(19) United States
(12) Patent Application Publication
(10) Pub. No.: US 2026/0175014 A1
(43) Pub. Date: Jun. 25, 2026

(54) POLYMER-BASED BUCKLING-RESISTANT INTRACORTICAL MICROELECTRODE ARRAY

(71) Applicant: University of Pittsburgh – Of the Commonwealth System of Higher Education, Pittsburgh, PA (US)

(72) Inventors: May Yoon Pwint, Pittsburgh, PA (US); Xinyan Cui, Wexford, PA (US)

(73) Assignee: University of Pittsburgh – Of the Commonwealth System of Higher Education, Pittsburgh, PA (US)

(21) Appl. No.: 19/127,656

(22) PCT Filed: Nov. 13, 2023
(86) PCT No.: PCT/US2023/079459

§ 371 Date: May 6, 2025

Related U.S. Application Data
(60) Provisional application No. 63/424,938, filed on Nov. 13, 2022.

(51) Int. Cl. A61N 1/05; H01B 3/30

(52) U.S. Cl. CPC A61N 1/0531; H01B 3/30

(57) ABSTRACT

Provided herein is a penetrative intracortical microelectrode array (MEA), comprising a body having a substantially T-shaped cross-sectional area, the body formed from an insulating biocompatible polymer surrounding individually addressable electrode traces connected to individually addressable electrode sites exposed to the exterior of the MEA. The MEA flexible yet is also buckling-resistant and can penetrate brain tissue without requiring secondary assistive methods.

### CROSS REFERENCE TO RELATED APPLICATIONS

This application claims priority to U.S. Provisional Application No. 63/424,938, filed Nov. 13, 2022, which is incorporated by reference in its entirety.

### ACKNOWLEDGMENT OF GOVERNMENT SUPPORT

This invention was made with government support under Grant No R01 NS110564 awarded by the National Institutes of Health. The government has certain rights in this invention.

### FIELD

This relates implantable microelectrode arrays for neurostimulation and recording, and related methods of manufacture.

### BACKGROUND

Nearly 1 in 6 of the world's population suffered from neurological disorders in 2007 according to the World Health Organization. The economic burden of neurological diseases was estimated to be $800 billion USD in 2017. Advances in neural engineering and neuroscience enable technologies for treatment of various nervous system disorders. One such enabling technologies is intracortical microelectrode arrays (MEAs).

Intracortical MEAs enable high resolution interfacing with the brain. However, implantation of such an MEA inevitably disrupts the native brain tissue, destroys the cells along the path of implant, ruptures the cell membranes and vasculature, and deforms the cells nearby. Moreover, tissue strain from device implantation also contribute to implantation injury and chronic tissue reaction around the implant. Intracortical MEAs fabricated on stiff substrates (for instance, silicon) cause higher mechanical strain on the tissue due to the mechanical mismatch between the implant and the brain tissue and continuously irritates the surrounding tissue through micromotion. On the other hand, flexible MEAs fabricated on polymers such as polyimide are mechanically compliant allowing them to move with the micromotions of the brain leading to better tissue integration. Therefore, for chronic implantation, flexible electrodes cause less strain on the surrounding tissue and soft material based electrodes are preferred for reduced immune reaction. However, soft and flexible electrodes are difficult to implant because they are mechanically compliant and buckle when inserting into the brain before penetrating through the pia mater. Many workaround strategies have been developed such as insertion shuttles, guides, stiffening coatings, shape memory polymers, magnetic guidance, and microfluidic actuation. However, the setups to apply those methods can be very cumbersome and often cause larger insertion trauma to the brain. Therefore, there is a need for flexible MEAs that can penetrate the brain without requiring secondary assistive methods.

### SUMMARY

Provided herein is a penetrative intracortical microelectrode array (MEA), comprising a body having a substantially T-shaped cross-sectional area, the body formed from an insulating biocompatible polymer surrounding individually addressable electrode traces connected to individually addressable electrode sites exposed to the exterior of the MEA. The MEA flexible yet is also buckling-resistant and can penetrate brain tissue without requiring secondary assistive methods.

In some examples, a penetrative intracortical microelectrode array (MEA) is provided, comprising a body having a substantially T-shaped cross-sectional area comprising a base and a stem, the body further comprising a proximal end and a distal end, and a longitudinal axis extending perpendicularly from the cross-sectional plane from the proximal end to the distal end. The base comprises a top and a bottom, with the stem extending from the top of the base; the width of the base is greater than the width of the stem, with flanges of the base extending beyond either side of the stem. The body is formed from an insulating biocompatible polymer (e.g., parylene C, polyimide, and SU-8) surrounding individually addressable electrode traces connected to individually addressable electrode sites exposed to the exterior of the MEA. The electrode traces are located within the stem of the MEA, and the electrode sites are located on the top of the flanges of the base. In some examples, the electrode traces and electrode sites are made of gold, platinum, iridium, indium tin oxide, conducting carbon, conducting polymer, or poly(3,4-ethylenedioxythiophene) (PEDOT). The distal end of the MEA is shaped for insertion into brain tissue and the proximal end is configured for connection to, or is connected to, an insertion device and also for electrical connection to an external neurostimulator or recording device for stimulating or recording neural signals from the electrode sites.

In some examples, a neural probe comprising one or more of the MEAs described herein is provided.

Also provided is a method of manufacturing the MEA provided herein. In some examples, the method comprises coating a SiO₂ surface of a silicon wafer with a first layer of the insulating biocompatible polymer, masking the first layer of insulating biocompatible polymer with a positive photoresist, alumina, or metal such as aluminum, titanium, and chromium in the shape of the base of the body of the microelectrode via lithography, and etching the masked first layer of insulating biocompatible polymer to form a first layer of the base. Next, the method comprises masking the first layer of the base with a positive photoresist in a pattern outlining the electrode sites and electrode connections between traces and electrode sites, photo-patterning and developing the masked first layer of the base, depositing a layer of conductive metal onto the photo-developed masked first layer of the base by metal evaporation, then stripping the positive photoresist to form a conductive metal containing first layer of the base. Next, the method comprises coating the conductive metal containing first layer of the base with a second layer of the insulating biocompatible polymer, masking the second layer of insulating biocompatible polymer with a positive photoresist, aluminum, or alumina, in a pattern outlining the shape of the base with electrode sites, and photo-developing and etching the masked second layer to form the base. Next, the method comprises repeating the coating, masking, photo-developing, depositing, and stripping steps to form the stem of the microelectrode containing electrode traces connected to the electrode sites in the base.

In another example, the method comprises coating a SiO₂ surface of a silicon wafer with a first layer of the insulating biocompatible polymer, masking the first layer of insulating biocompatible polymer with a photoresist, an oxide, a nitride, or metal such as aluminum, titanium, and chromium in the shape of the flange. Next, the method comprises photo-patterning a positive photoresist outlining the electrode sites and electrode connections between traces and electrode sites and developing the masked electrical outlines, depositing a layer of conductive metal onto the photo-developed masked first layer by metal evaporation, then stripping the positive photoresist to form a conductive metal containing first conductive layer. Next, the method comprises repeating the coating, masking, photo-developing, depositing, and stripping steps to form the stem of the microelectrode containing multiple layers of electrode sites and traces.

The foregoing and other features of this disclosure will become more apparent from the following detailed description of several aspects which proceeds with reference to the accompanying figures.

### BRIEF DESCRIPTION OF THE FIGURES

FIGS. 1A-1C: Three perspectives of an exemplary MEA. FIG. 1A is a three-quarter view.

FIG. 1B is a top view. FIG. 1C is a cross-sectional view. Not to scale.

FIG. 2: A perspective view of an exemplary MEA design showing a T-shaped cross section, not to scale.

FIG. 3A-D: Possible buckling scenarios during MEA insertion into brain tissue. FIG. 3A depicts the most likely scenario with proper surgical setup. FIG. 3B depicts a less likely scenario that could happen if there is movement due to a loose setup. FIG. 3C depicts the least likely scenario, which indicates improper setup. FIG. 3D is a schematic of a rat on a stereotaxic frame illustrating the surgical setup.

FIG. 4 shows a graph of dimension calculations for MEAs as provided herein.

FIG. 5A. Exemplary process for MEA fabrication. a) Spin coat polyimide precursor (e.g., PI2525) on a clean Si/SiO₂ wafer. b) Spin coat positive photoresist (e.g., AZ P4620), photopattern it with a maskless aligner, and cure with developer (e.g., AZ400K). c) Dry etch the polyimide with O2 and SF6 in a reactive ion etcher, remove the positive photomask in acetone, and cure in N2. d) Spin coat another polyimide layer, photopattern, and develop. e) Evaporate a layer of Au using an electron beam evaporator machine. f) Lift off the sacrificial mask in acetone to reveal the Au patterns. g) Spin coat another polyimide layer, photopattern a mask, dry etch the polyimide layer, remove the mask, and cure the polyimide. h) Repeat steps d-g with appropriate masks designed beforehand. Illustration not to scale.

FIGS. 5B and 5C. Another exemplary process for MEA fabrication. a) Spin coat polyimide precursor (e.g., PI2525) on a clean Si/SiO₂ wafer. b) Spin coat positive photoresist (e.g., AZ P4620), photopattern it with a maskless aligner, and cure with developer (e.g., AZ400K). c) Evaporate a metal layer (e.g., Au or Pt) using an electron beam evaporator machine, and lift off the sacrificial mask. d) Apply a hard mask covering the flange area of the MEA. e) Spin coat positive photoresist and cure with developer. f) Spin coat positive photoresist, photopattern it with a maskless aligner, cure with developer, evaporate a metal layer (e.g., Au or Pt) using an electron beam evaporator machine, and lift off the sacrificial mask. g) Spin coat positive photoresist and cure with developer. h) Apply a hard mask covering the stem area of the MEA. i) Apply reactive ion etching (RIE) and release the hard mask to obtain the finished MEA. Illustration not to scale.

FIG. 6. A perspective view of an MEA as provided herein where the proximal (or “back”) end of the MEA is connected to a printed circuit board (PCB) containing linkages to each electrode trace in the MEA. The PCB connected to the MEA serves as an insertion device and also contains electrical connectors that couple to the electrode traces in the stem of the MEA.

FIGS. 7A and 7B show images of an MEA as provided herein.

FIGS. 8A-8E. An image of an MEA as provided herein is shown in FIG. 8A. The cross-sectional height of the MEA along the dashed line shown in FIG. 8A is provided in the graph shown in FIG. 8B. A schematic diagram of a cross section of the MEA with dimensions indicated is shown in FIG. 8C. FIG. 8D shows a scanning electron microscope image of the MEA. The impedance (Z) of the MEA relative to stimulation frequency is shown in FIG. 8E.

FIG. 9A-9C: Results of finite element analysis performed using Autodesk Fusion 360 comparing von Mises strain caused by T-beam (FIG. 9A), flat polyimide (FIG. 9B), and silicon (FIG. 9C) MEAs.

FIG. 10A-10B: In vitro test insertion into gel of a flat/rectangular polyimide MEA or T-shaped MEA as provided herein (FIG. 10A) and compiled results (FIG. 10B).

FIG. 11A-11C: In vivo insertion of an MEA as provided herein into rat brain. FIG. 11A shows a perspective view of an inserted probe in rat brain; FIG. 11B shows a close-up view of the insertion point. The MEA was inserted repeatedly into the brain tissue, and results for successful insertion (without buckling) for the first and second insertions are shown in FIG. 11C.

### DETAILED DESCRIPTION

Although many assistive technologies have been designed to facilitate the implantation of flexible neural probes, there has been virtually no work done in terms of the probe design itself to improve insertion of penetrative neural interfaces. The penetrative intracortical microelectrode array (MEA) provided herein is the first polymer-based flexible penetrating MEA to be implanted without additional assistive devices. This allows for minimal insertion trauma and small implant footprint. Furthermore, due to simplicity of the surgical procedure made possible using the provided MEA, insertion of multiple MEAs during the same surgery is possible.

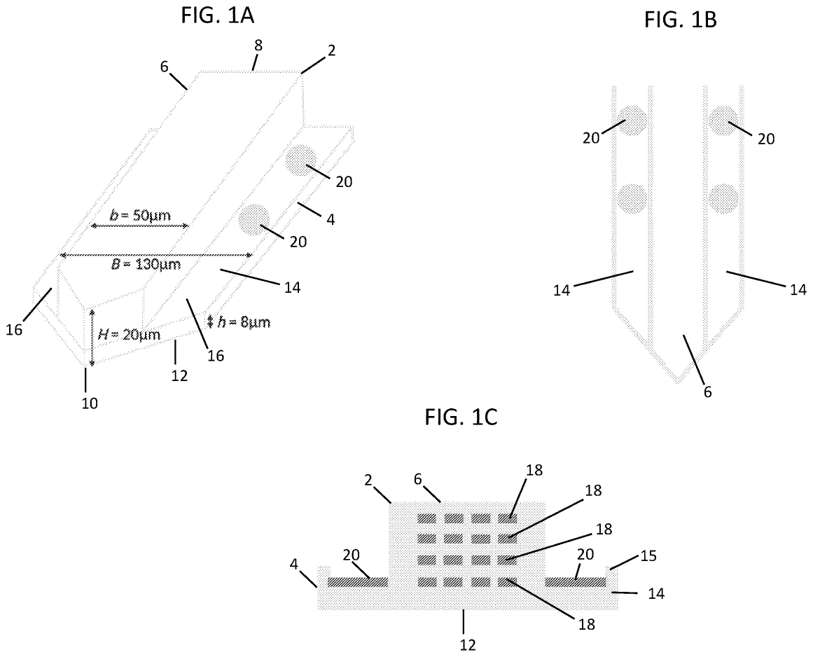

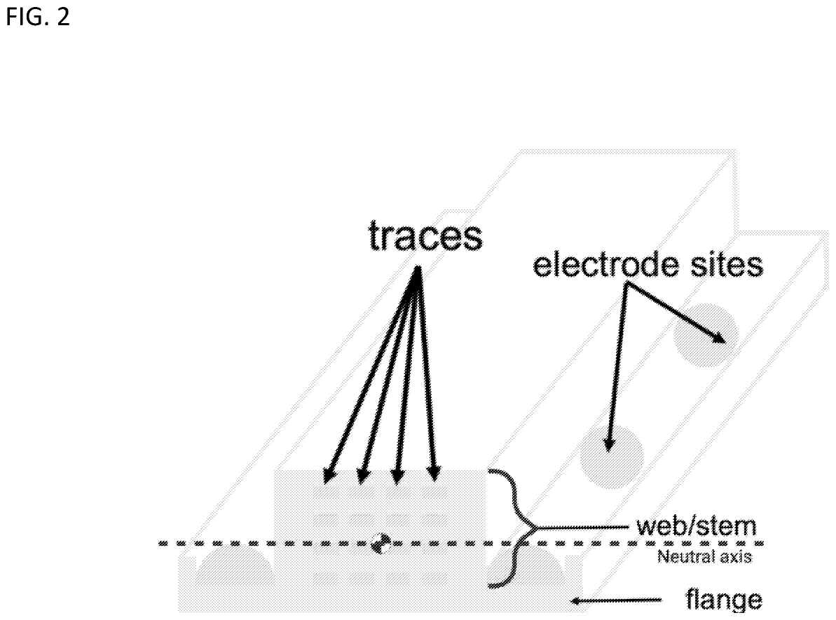

As discussed in more detail below, the MEA is substantially T-shaped. By leveraging the mechanics of T-beams, the MEA has higher buckling strength for the same cross-sectional area compared to MEAs with rectangular cross section. The T-shaped cross section takes advantage of resistance of vertical stem to bending moment by shifting the neutral axis outside the flat flange area (see FIG. 2). Further, the electrode sites are designed on the sides and the conducting traces of the electrodes are stacked in layers in the vertical stem portion of the T (See FIG. 1C), thereby increasing the number of channels per MEA width compared to a typical flat design, while maintaining buckling strength.

Thus, the MEA provided herein is resistant to buckling during insertion into brain tissue. Further, the MEA is insertable into brain tissue without support provided by a secondary solid substrate, or the assistance of a shuttle, a secondary stiff coating, or an insertion device that supports the MEA from buckling during insertion. The flexible nature of the MEA creates a less acute and chronic immune reaction upon insertion than a standard silicon Michigan electrode array. In some examples, the MEA does not buckle when inserted into brain tissue with a force of 1 mN or less.

FIG. 1A shows a perspective view of an example of an MEA as provided herein. As shown, the MEA has a body 2 having a substantially T-shaped (or inverted T-shape, as shown in the figure) cross-sectional area comprising a base 4 and a stem 6. The body further comprises a proximal end 8 and a distal end 10. A longitudinal axis extends perpendicularly from the cross-sectional plane from the proximal end to the distal end. The base comprises a bottom 12 and a top 14, with the stem extending from the top of the base.

The width of the base B is greater than the width of the stem b. In the figure, the values for B and b are shown as 130 μm and 50 μm, respectively; however the values for B and b can vary according to the parameters of the MEA as provided herein. Due to the narrower width of the stem compared to the base, the base extends beyond either side of the stem, forming a flange 16 on either side of the stem.

The flanges on either side of the stem need not be symmetrical, though symmetrical implementations are also provided. In some implementations, the stem is centered on the base such that each flange is about the same width. FIG. 1B shows a top view of an implementation of the MEA with flanges 14 that are about the about the same width.

The body 2 is formed from an insulating (that is, non-conductive) biocompatible polymer surrounding individually addressable electrode traces 18 connected to individually addressable electrode sites 20 exposed to the exterior of the MEA.

FIG. 1C shows a cross-sectional view of the body 2 of the MEA, illustrating the electrode traces 18 in the stem 6. The electrode traces 18 travel in the stem 6 from the proximal end 8 of the MEA towards the electrode sites 20, and are electrically connected to the electrode sites 20. Typically, each trace in the stem is connected to a single electrode site, although in some implementations multiple traces are connected to a single electrode site. Thus, the number of traces in the stem of the microelectrode can vary based on the number of electrode sites. As shown in FIG. 1C, the electrode traces 18 have a cross-sectional shape that is rectangular; however, any suitable shape may be used, such as round, oval or square. A feature of the MEA described herein is the stacking of traces vertically in the stem portion of MEA, which allows maximization of channel count while keep the total width of the stem constant. Additionally, any suitable configuration may be used to connect the traces 18 to the electrode sites 20. Typically, the traces closest to the base will connect through the stem and/or base to the electrode sites closest to the proximal end, the traces in the next stem layer farther from the base will connect through the stem and/or base to the next set of electrode sites, and so on.

As depicted in FIG. 1C, in some examples, the base comprises lips 15 at the outer edges of the flanges of the base, and which extend above the top of the base and parallel to the stem. The height of the lips above the top of the base typically is no more than the height of the base itself, for example, from 5% to 50% (such as 5% to 40%, from 5% to 30%, from 5% to 20%, from 5% to 10%, from 10% to 50%, from 10% to 40%, from 20% to 50%, or from 30% to 50%) of the height of the base.

The insulating biocompatible polymer can be any suitable polymer that is sufficiently flexible to provide the MEA a level of elasticity that minimizes injury or inflammation in surrounding tissue when the MEA is implanted in the brain, and also provides the MEA with sufficient stiffness for insertion into brain tissue without buckling, for example with an insertion force of 2 mN or less, such as 1 mN or less. In some implementations, the insulating biocompatible polymer is parylene C, polyimide, or SU-8.

The electrode traces, electrode sites, and any connection between the two, are made of any material suitable for conducting an electrical signal through the MEA either from a stimulator to the surrounding brain tissue or from the surrounding brain tissue to a recording device. In some implementations, the electrode traces, electrode sites, and any connection between the two, are made of are made of gold, platinum, iridium, indium tin oxide, or a conducting carbon or polymer, such as poly(3,4-ethylenedioxythiophene) (PEDOT).

The electrode sites 20 are located on the top of the base on the flanges of the base. The number of electrode sites on the MEA can vary as needed depending on the intended use of the MEA. In some implementations, the MEA comprises from 6 to 128 individually addressable electrode sites, such as from 6-62 electrode sites, or 6-32 electrode sites, or 8, 16, 32, 64, or 128 individually addressable electrode sites.

The positioning of the electrode sites 20 on the flanges 16 of the MEA and the spacing between electrode sites on each flange can vary as needed depending on the intended use of the MEA. In some implementations, the electrode sites are evenly distributed on each flange of the base. In some implementations, the electrode sites are evenly distributed within the distal half of the MEA. In some implementations, the electrode sites are evenly distributed along the entire length of the shank of the MEA.

The electrode sites 20 can have any size suitable for conducting an electrical signal through the MEA either from a stimulator to the surrounding brain tissue or from the surrounding brain tissue to a recording device. Additionally, the electrode sites individually can have any shape that is suitable for this purpose, such as round, oval, polygonal, square or rectangular. In some implementations, the electrode sites individually have a top surface area of from 100-10,000 μm².

The distal end 10 is shaped for insertion into brain (e.g., cortical) tissue, for example, with a rounded or sharp point at the distal tip of the distal end 10. The proximal end 8 is configured for connection to, or is connected to, heterologous equipment such as an insertion device and/or an electrical connection (such as one or more leads) connecting to external neurostimulator or recording device.

The overall size of the MEA can vary as needed depending on the intended use of the MEA. In some implementations, the length of the body along the longitudinal axis is from 2-10 mm, the width of the base is from 30-140 μm, the width of the stem is from 15-60 μm, the height of the body from the bottom of the base to the top of the stem is from 14-42 μm; and the height of the base from the bottom of the base to the top of the base is from 5-14 μm. In a non-limiting implementation, the length of the MEA along the longitudinal axis is about 2 mm, the width of the base is about 130 μm, the width of the stem is about 50 μm, the height of the body from the bottom of the base to the top of the stem is about 20 μm, and the height of the base from the bottom of the base to the top of the base is about 8 μm. In another non-limiting implementation, the length of the MEA along the longitudinal axis is about 2 mm, the width of the base is about 125 μm, the width of the stem is about 60 μm, the height of the body from the bottom of the base to the top of the stem is about 15 μm, and the height of the base from the bottom of the base to the top of the base is about 5 μm.

In some implementation, the MEA can be used as a single multi-channel electrode for insertion into brain tissue and recording and/or stimulating neural signals. In other implementations, an array is provided that comprises two or more of the MEAs as provided herein. Any suitable number of the MEA as provided herein can be included in the array, depending on the intended use of the array.

During use, the MEA is typically coupled to recording and/or stimulating circuitry. In some implementations, for example for laboratory use, the microelectrode is implanted into brain tissue and the recording and/or stimulating circuitry is external. In some implementations, the circuitry can be integrated circuitry that is fully implanted (typically implantable in a subcutaneous pocket within a patient's body) or partially implanted in the patient. Coupling of the circuitry to the microelectrode can be by way of one or more leads, although any operable coupling capable of transmitting the measured neural signal from the electrode sites of the MEA to the circuitry, or a stimulation signal from the circuitry to the electrodes, can be used.

Also provided is a method of manufacturing the MEA provided herein. The method comprises coating a SiO₂ surface of a silicon wafer with a first layer of the insulating biocompatible polymer, masking the first layer of insulating biocompatible polymer with a positive photoresist, aluminum, or alumina in the shape of the base of the body of the microelectrode via lithography, and etching the masked first layer of insulating biocompatible polymer to form a first layer of the base. The first layer of the base is then masked with a positive photoresist in a pattern outlining the electrode sites and electrode connections between traces and electrode sites. Photo-patterning and developing of the masked first layer of the base is then performed, followed by depositing a layer of conductive metal onto the photo-developed masked first layer of the base by metal evaporation, then stripping the positive photoresist to form a conductive metal containing first layer of the base. The conductive metal containing first layer of the base is coated the with a second layer of the insulating biocompatible polymer, followed by masking the second layer of insulating biocompatible polymer with a positive photoresist, aluminum, or alumina, in a pattern outlining the shape of the base with electrode sites, and photo-developing and etching the masked second layer to form the base. The coating, masking, photo-developing, depositing, and stripping steps are repeated to form the stem of the microelectrode containing electrode traces connected to the electrode sites in the base.

Unless otherwise noted, technical terms are used according to conventional usage. As used herein, the term “comprises” means “includes.” Although many methods and materials similar or equivalent to those described herein can be used, particular suitable methods and materials are described below. In addition, the materials, methods, and examples are illustrative only and not intended to be limiting. The scope of the claims should not be limited to those features exemplified. To facilitate review of the various implementations, the following explanations of terms are provided:

Unless context indicated otherwise, the term “about” refers to plus or minus 5% of a reference value. For example, “about” 100 refers to 95 to 105.

The term “electrical stimulus” refers to the passing of various types of current or voltage selectively through one or more electrodes of a device to or from a target location in a subject.

The terms “electrode site” and “trace” refer to an electric conductor through which an electric current can pass. An electrode site is exposed to the surrounding area (e.g., neural tissue) of the electrode-containing device. A trace is embedded within a non-conductive material and connects the electrode site to secondary aspects of the device. An electrode site connected to one or more traces can be a collector and/or emitter of an electric current. Non-limiting examples of conducting materials for electrode sites and traces include noble metals and alloys, such as stainless steel and tungsten.

The term “neural probe” refers to a device or component of a device including one or more electrodes that can be placed in contact with neuronal tissue (e.g., in an animal host) and can record and/or stimulate neural signals from or to the neuronal tissue. Neural probes typically include conductive and non-conductive surfaces designed for contact with neuronal tissue when implanted at a target location in a subject, and can include one or more electrodes that can be independently monitored from other conductive surfaces on or off the probe) for recording current at the target location.

The term “neural signal” refers to an electrical signal originating in the nervous system of a subject. “Stimulating a neural signal” refers to application of an electrical current to the neural tissue of a subject in such a way as to cause neurons in the subject to produce an electrical signal (e.g., an action potential). An extracellular electrical signal can, however, originate in a cell, such as one or more neural cells. An extracellular electrical signal is contrasted with an intracellular electrical signal, which originates, and remains, in a cell. An extracellular electrical signal can comprise a collection of extracellular electrical signals generated by one or more cells.

The term “neurostimulator” refers to a current or voltage-controlled electrical stimulation device. A neurostimulator controls the delivery of an electrical pulse, or pattern of electrical pulses, having defined parameters, for example and without limitation, pulse frequency, duration, amplitude, phase symmetry, duty cycle, pulse current, pulse width, and on-time and off-time. The controlled electrical pulse is delivered through one or more electrodes configured to apply the electrical stimulus to the brain of a subject. Neurostimulators may be utilized to apply a series of electrical pulse stimuli (e.g., charge balanced pulses) through at least one electrode; for example and without limitation, low-frequency pulse train patterns, frequency-sequenced pulse burst train patterns (e.g., wherein different sequences of modulated electrical stimuli are generated at different burst frequencies), and phasic train patterns (e.g., wherein the stimulus control parameters change over the course of feedback, from a distal source).

As used herein, the term “coupled” generally means physically coupled or linked and does not exclude the presence of intermediate elements between the coupled items absent specific contrary language.

### Additional Description

Clause 1. A penetrative intracortical microelectrode array (MEA), comprising:
a body having a substantially T-shaped cross-sectional area comprising a base and a stem, the body further comprising a proximal end and a distal end, and a longitudinal axis extending perpendicularly from the cross-sectional plane from the proximal end to the distal end, and wherein:
the base comprises a top and a bottom, with the stem extending from the top of the base,
the width of the base is greater than the width of the stem, with flanges of the base extending beyond either side of the stem;
the body is formed from an insulating biocompatible polymer surrounding individually addressable electrode traces connected to individually addressable electrode sites exposed to the exterior of the MEA;
the electrode traces are located within the stem of the MEA;
the electrode sites are located on the top of the flanges of the base;
the distal end is shaped for insertion into brain tissue and the proximal end is configured for connection to, or is connected to, an insertion device and also for electrical connection to an external neurostimulator or recording device for stimulating or recording neural signals from the electrode sites.
Clause 2. The MEA of clause 1, wherein the insulating biocompatible polymer is selected from parylene C, polyimide, and SU-8.
Clause 3. The MEA of clause 1 or clause 2, wherein the electrode traces and electrode sites are made of gold, platinum, iridium, indium tin oxide, conducting carbon, conducting polymer, or poly(3,4-ethylenedioxythiophene) (PEDOT).
Clause 4. The MEA of any one of the prior clauses, wherein the distal end of the body has a pointed shape to facilitate penetration into cortical tissue.
Clause 5. The MEA of any one of the prior clauses, comprising from 6 to 128 individually addressable electrode sites.
Clause 6. The MEA of any one of the prior clauses, comprising 8, 16, or 32 individually addressable electrode sites.
Clause 7. The MEA of any one of the prior clauses, wherein the electrode sites individually have a surface area of from 100-10,000 μm².
Clause 8. The MEA of any one of the prior clauses, wherein the electrode sites are evenly distributed on each flange of the base.
Clause 9. The MEA of any one of clauses 1-7, wherein the electrode sites are evenly distributed within the distal half of the microelectrode and on the top of the flanges of the base.
Clause 10. The MEA of any one of the prior clauses, wherein the length of the body along the longitudinal axis is from 2-10 mm;
the width of the base is from 30-140 μm;
the width of the stem is from 15-60 μm;
the height of the body from the bottom of the base to the top of the stem is from 14-42 μm; and
the height of the base from the bottom of the base to the top of the base is from 5-14 μm.
Clause 11. The MEA of any one of the prior clauses, wherein
the length of the body along the longitudinal axis is about 2 mm;
the width of the base is about 130 μm;
the width of the stem is about 50 μm;
the height of the body from the bottom of the base to the top of the stem is about 20 μm; and
the height of the base from the bottom of the base to the top of the base is about 8 μm.
Clause 12. The MEA of any one of the prior clauses, wherein the stem is centered on the base such that the flanges of the base extending beyond either side of the stem are about the same width.
Clause 13. The MEA of any one of the prior clauses, wherein the microelectrode does not buckle when inserted into brain tissue with a force of 1 mN or less.
Clause 14. The MEA of any one of the prior clauses, wherein microelectrode is not supported by a secondary solid substrate.
Clause 15. The MEA of any one of the prior clauses, wherein the microelectrode is insertable into brain tissue without assistance of a shuttle, a secondary stiff coating, or an insertion device that supports the microelectrode from buckling during insertion.
Clause 16. A neural probe, comprising one or more of the MEA of any one of the prior clauses.
Clause 17. A method of manufacturing the MEA of any one of clauses 1-15, comprising:
coating a SiO₂ surface of a silicon wafer with a first layer of the insulating biocompatible polymer, masking the first layer of insulating biocompatible polymer with a positive photoresist, aluminum, or alumina in the shape of the base of the body of the microelectrode via lithography, and etching the masked first layer of insulating biocompatible polymer to form a first layer of the base;
masking the first layer of the base with a positive photoresist in a pattern outlining the electrode sites and electrode connections between traces and electrode sites, photo-patterning and developing the masked first layer of the base, depositing a layer of conductive metal onto the photo-developed masked first layer of the base by metal evaporation, then stripping the positive photoresist to form a conductive metal containing first layer of the base;
coating the conductive metal containing first layer of the base with a second layer of the insulating biocompatible polymer, masking the second layer of insulating biocompatible polymer with a positive photoresist, aluminum, or alumina, in a pattern outlining the shape of the base with electrode sites, and photo-developing and etching the masked second layer to form the base;
repeating the coating, masking, photo-developing, depositing, and stripping steps to form the stem of the microelectrode containing electrode traces connected to the electrode sites in the base.

### EXAMPLES

The following examples are provided to illustrate particular features of certain aspects of the disclosure, but the scope of the claims should not be limited to those features exemplified.

### Example 1

### Intracortical MEA Design

This example illustrates design considerations such as methods by which intracortical MEAs may be inserted and potential points of failure in intracortical MEA insertion. This example explains how these considerations affect one exemplary intracortical MEA design.

Intracortical MEAs must withstand approximately 1 mN force to successfully penetrate the rat brain after durotomy. W. Jensen et al., Proc. 25th Ann. Int. Con. IEEE Eng. in Med. and Bio. Soc. (2003) 3:2168-71. This example illustrates design of an MEA that can support at least 1 mN buckling load with maximum number of electrode sites on the probe.

A major challenge with implantation of flexible probes is structural buckling. Buckling of a flexible probe can be modeled by Euler buckling equation, given as Equation 1:

$${F}_{B} = \frac{{π}^{2} EI}{{( KL )}^{2}} \qquad ( 1 )$$

F_{B}=critical buckling force,
E=modulus of elasticity; Young's modulus,
I=the moment of inertia,
L=the length of the probe, and
K=a coefficient that depends on end conditions; effective length factor.

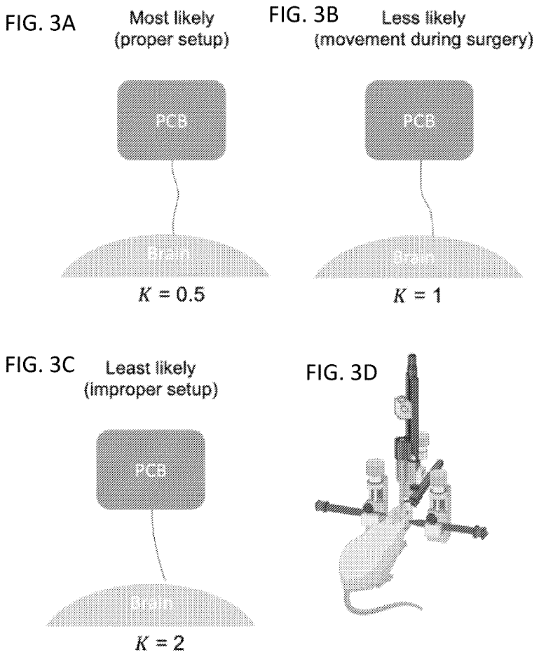

During implantation of penetrative microelectrodes, the “proximal end” is fixed to a stereotaxic inserter and the “distal end” should touch the brain surface before penetration (FIG. 1). When the insertion force is applied to the probe, there are three possible buckling scenarios giving rise to three different K values. Since the proximal end is affixed to a stereotaxic frame, no rotation or lateral translation can occur at the fixed end. According to the surgical setup described above, the most likely scenarios is that the penetrating/distal end will be translation fixed and rotation fixed (FIG. 3A) since it will be touching the brain right before the start of insertion. If, however, the electrode does not touch the brain surface, scenario in FIG. 3C would be more representative of the situation. The scenario in FIG. 3B could happen if movement happens while the electrode is touching the brain surface during insertion. Therefore, proper surgical setup is crucial for successful insertion and surgery will proceed only if everything is setup correctly. Thus, K=0.5 is assumed for design purposes.

Related to the coefficient K is the length of the probe. According to Equation 1, the critical load decreases with the square of the length. Therefore, shank length of 2 mm is chosen to constrain the design space. Since in this example an intracortical electrode for a rat model is designed, a 2 mm long shank is sufficient.

As seen in Equation 1, another important design parameter is modulus of elasticity (E) which is a fundamental property of materials. To simplify the calculations and design considerations, it was assumed that the modulus of the proposed probe is governed by that of the insulator material, since there are more insulation layers than conductive ones and each insulating film is on the order of micrometers while a conductive layer is on the order of nanometers. Non-limiting examples of biocompatible polymer-based insulators suitable for neural interfaces include polyimide, parylene-C, and SU8. Although Young's modulus is a fundamental property, varying results have been reported for these materials. Young's modulus can be estimated from nanoindentation tests. Among the three biocompatible polymers mentioned, polyimide has the highest modulus of elasticity (Table 1). Therefore, in one example, polyimide is chosen as the substrate material for the intracortical electrode.

TABLE 1
Young's moduli of SU-8, Parylene-C, and Polyimide

| Material | Modulus (GPa) |
|---|---|
| SU8 | 0.0059508 |
| Parylene C | 4.723574 |
| Polyimide | 5.3939534 |

Using E=5.39 GPa, K=0.5, L=2 mm, and F_{B}=1 mN, the minimum required moment of inertia was computed using Equation 1. For a T cross-section probe, the moment of inertia depends on the width of the flange (B), the width of the web (b), the total thickness (H), the thickness of the stem (h), and the location of the centroid and can be calculated from the formula of Equation 2:

$$I = \frac{{Bh}^{3}}{12} + {Bh ( {y}_{c} - \frac{h}{2} )}^{2} + \frac{{b ( H - h )}^{3}}{12} + b ( H - h ) {( {y}_{c} - \frac{H + h}{2} )}^{2} \qquad ( 2 )$$

$${y}_{c} = [ \frac{h}{2} ( Bh ) + \frac{H - h}{2} \cdot b ( H - h ) ] / [ Bh + b ( H - h ) ]$$

B=width of the flange
b=width of the web
H=total thickness
h=thickness of the stem

Based on these calculations, an MEA was designed with a length L of 2 mm, a base width B of 130 μm, a stem width b of 50 μm, an overall height H of 20 μm, and a base height h of 8 μm. See FIG. 1.

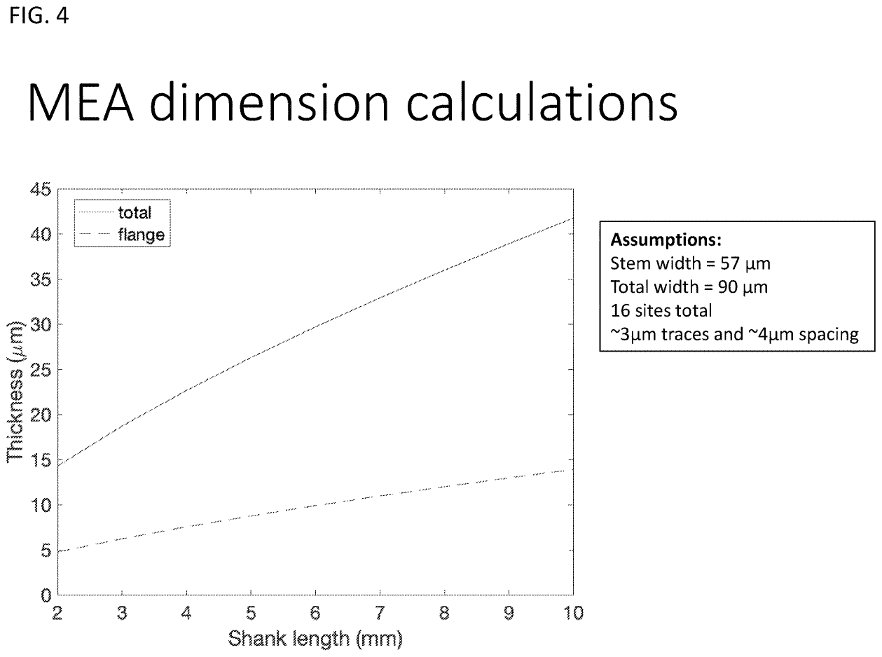

Calculations for MEA thickness, including total height (from the bottom of the base to the top of the stem) and flange height (from the bottom to the top of the flange) based on shank length are shown in FIG. 4. For the calculations in the figure, it is assumed that the stem width is 57 μm, the base/total width is 90 μm, and that the MEA includes 16 electrode sites, 8 on each flange evenly spaces, with traces ˜3 μm in cross-sectional diameter, and with 4 μm spacing.

### Example 2

### Intracortical MEA Microfabrication

This example illustrates an exemplary process of fabricating the MEA designed in Example 1.

Once B, b, H, and h are determined, masks for probe fabrication can be designed in silico using computer aided design (CAD) software such as KLAYOUT®. The MEA was fabricated on a Si wafer with 100 μm thick SiO₂ layer.

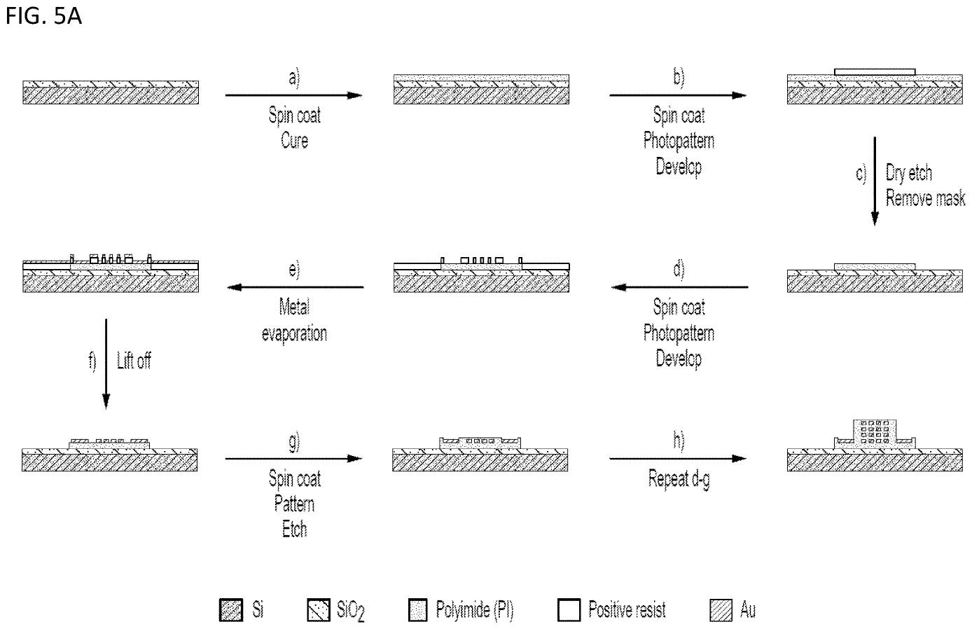

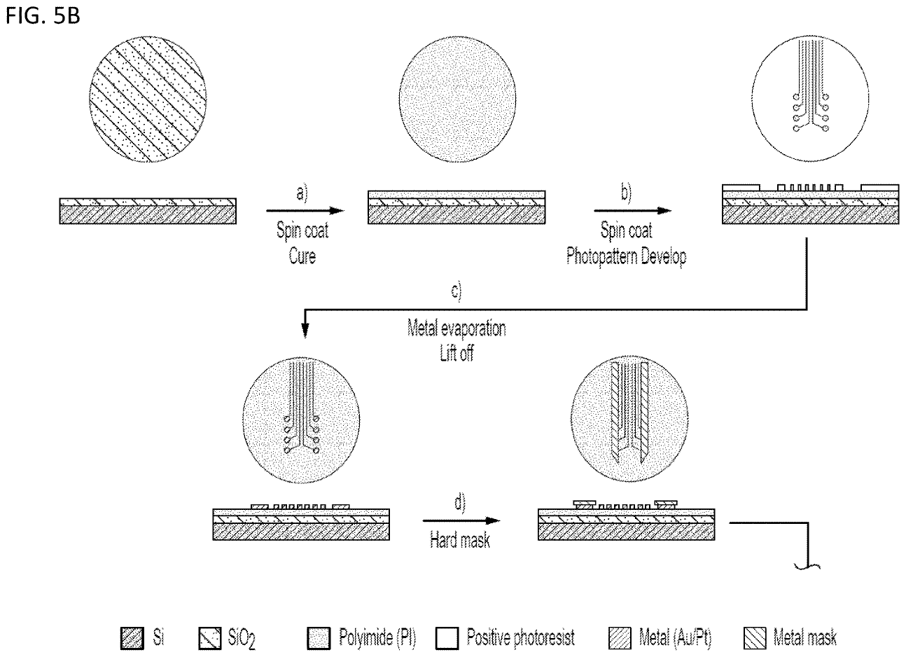

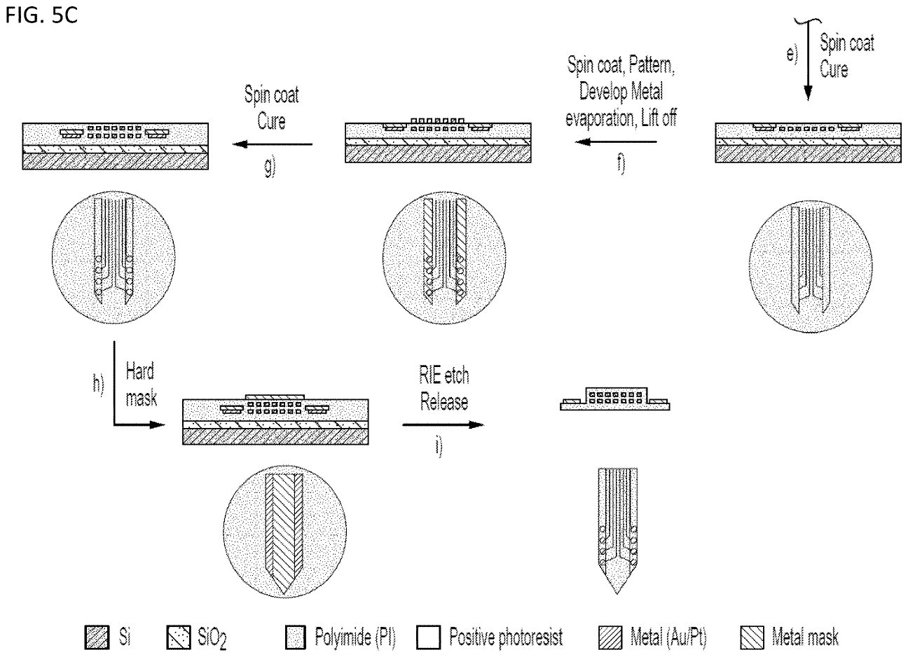

FIG. 5A illustrates a fabrication process used to generate the MEA. The Si wafer was first cleaned in acetone, isopropanol, and water, followed by an O₂ plasma. Non-photodefinable polyimide precursor PI2525 (HD Microsystems) was static dispensed and spin coated on the clean wafer. The precursor was then cured with N₂ flow in a tube furnace. Next, a positive photoresist was used to define a sacrificial photomask on top of the PI2525 layer using a maskless aligner. The photopatterned positive resist was developed via wet etching and PI2525 was dry etched using O₂ and SF₆. The remaining photomask was stripped off in acetone and a new layer of positive photoresist was spin coated, patterned, and developed to pattern the first metal layer. A layer of gold (Au) was evaporated onto the patterned sacrificial photomask. Polyimide and Au layers were alternated using the spin coating, etching, and evaporation methods until the desired total thickness was achieved. To maximize the channel count, the Au traces were stacked vertically in the stem portion of the T on the alternating layers. Thus, a feature of the MEA design described herein is the stacking of traces vertically in the stem portion of the MEA on the alternating layers which allows maximization of channel count while keep the total width constant. Finally, the device was released by lifting off in buffered oxide etch (BOE) overnight.

FIGS. 5B and 5C illustrate another fabrication process used to generate the MEA. The Si wafer was first cleaned in acetone, isopropanol, and water, followed by an O₂ plasma. Non-photodefinable polyimide precursor PI2525 (HD Microsystems) was static dispensed and spin coated on the clean wafer. The precursor was then cured with N₂ flow in a tube furnace. Next, a positive photoresist was used to define a sacrificial photomask on top of the PI2525 layer using a maskless aligner. The photopatterned positive resist was developed via wet etching and a layer of metal suitable for the electrodes and traces of the MEA (e.g., Au or Pt) was evaporated onto the patterned sacrificial photomask, and the sacrificial mask then lifted off in suitable solvent such as acetone to reveal the metal patterns. A hard metal mask was then applied to cover the flange area of the MEA. Next, a new layer of polyimide was coated and cured. A new layer of positive photoresist was spin coated, patterned, and developed to pattern the second metal layer, which was then evaporated onto the patterned sacrificial photomask, and the sacrificial mask then lifted off in acetone to reveal the metal patterns. A further layer of polyimide was spin coated and cured, after which a hard metal mask applied to the stem region of the MEA. Finally, reactive ion etching (RIE) was used remove excess polyimide, and the hard mask and sacrificial SiO₂ layer were etched to obtain the finished MEA.

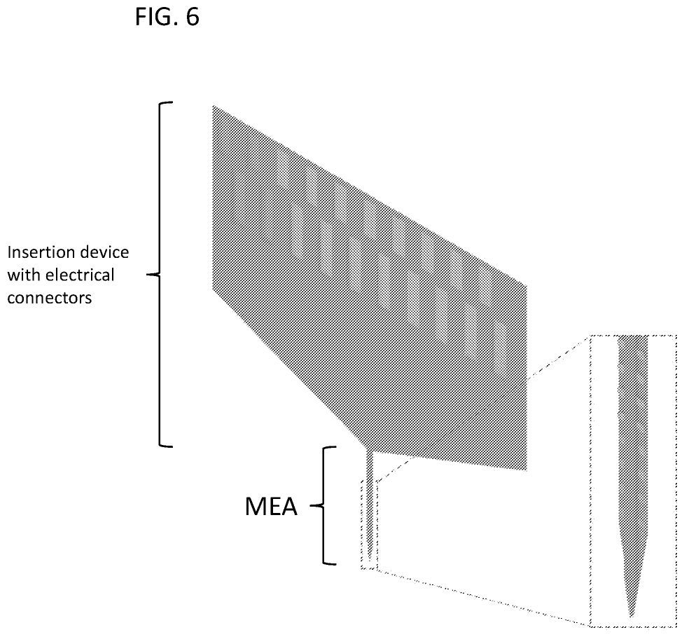

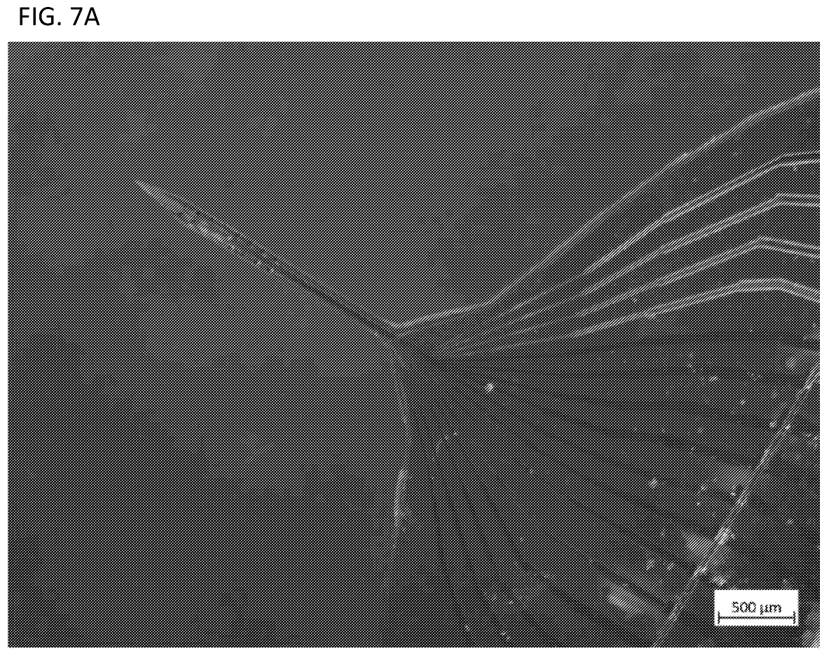

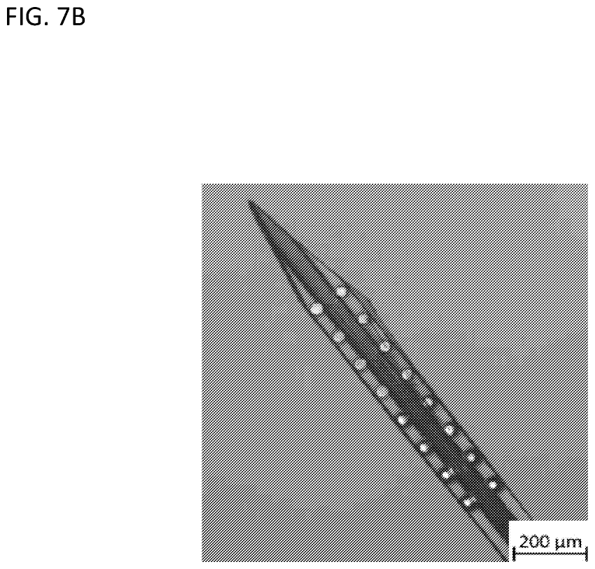

FIG. 6 shows a diagram of designed MEA where the proximal (or “back”) end of the MEA is connected to a solid support that functions both as an insertion device and also contains electrical connectors that link to the electrode traces in the stem of the MEA. FIGS. 7A and 7B show images of the manufactured device. In FIGS. 6 and 7, microfabricated electrode (with dimensions of the shank approximately set forth as a length of 2 mm, the width of the base about 130 μm, the width of the stem about 50 μm, the height of the body from the bottom of the base to the top of the stem about 20 μm, and the height of the base from the bottom of the base to the top of the base about 8 μm) was inserted into the zero-insertion force (ZIF) connector of a printed circuit board (PCB) containing linkages to each electrode trace in the MEA. The PCB was attached to a stereotaxic arm whose speed was controlled by a piezo motor.

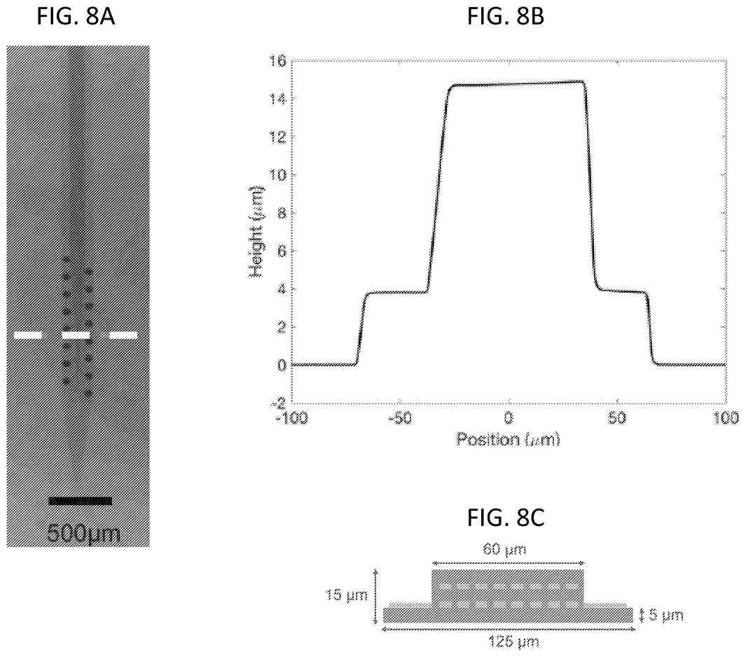

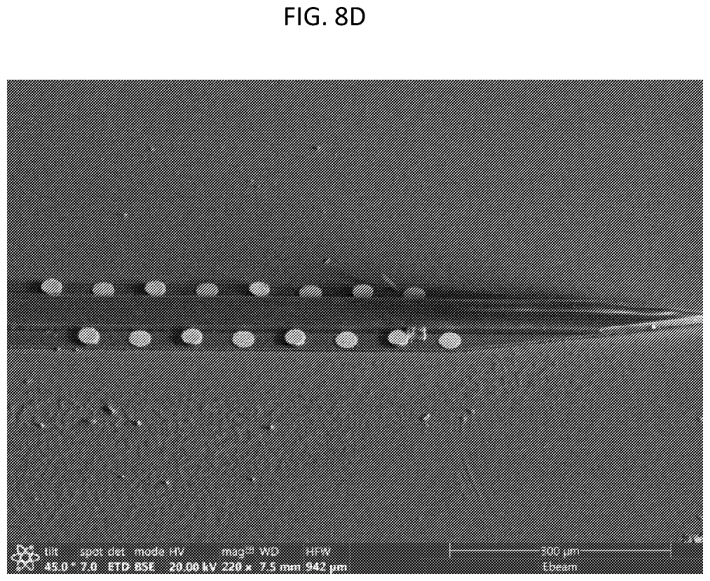

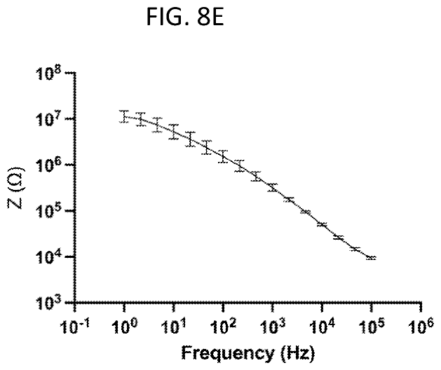

FIGS. 8A and 8D show images of another MEA manufactured using the method provided herein. The cross-sectional height of the MEA along the dashed line shown in FIG. 8A is provided in the graph shown in FIG. 8B. A schematic diagram of a cross section of the MEA with dimensions indicated is shown in FIG. 8C. The electrical properties of the device were assessed, and the impedance (Z) of the MEA relative to stimulation frequency is shown in FIG. 8E.

### Example 3

### Intracortical MEA Assessment

This example illustrates improved buckling resistance by T-beam probes.

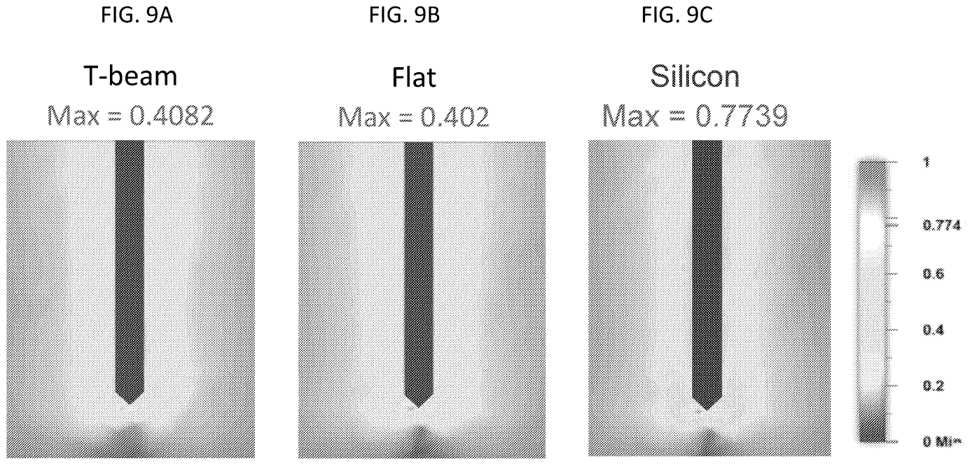

First, finite element analysis was done in Autodesk Fusion 360 to compare the tissue strain caused by the T-beam MEAs of produced in Example 2 (FIG. 9A) to conventional flat/rectangular polyimide (FIG. 9B) and stiff silicon probes (FIG. 9C). The results show that even though T-beams have much higher buckling strength, they pose virtually no additional strain on the surrounding brain tissue than flat polyimide probes. Compared to stiff silicon probes, the maximum equivalent strain a MEA as provided herein causes is almost 50% less than the current “gold standard” silicon probe.

Next, to assess the enhanced stability of the T-beam design compared to flat/rectangular MEA designs, a flat/rectangular MEA made of polyimide was compared with the MEAs produced according to the method of Examples 1 and 2, by insertion into a gel matrix.

Two microfabricated probes were assessed, one with the dimensions should in FIG. 1A and another with the dimensions shown in FIG. 8C. The first (Probe 1) had dimensions approximately set forth as a length of 2 mm, the width of the base about 130 μm, the width of the stem about 50 μm, the height of the body from the bottom of the base to the top of the stem about 20 μm, and the height of the base from the bottom of the base to the top of the base about 8 μm, with 16 electrode sites. The second (Probe 2) had dimensions approximately set forth as a length of 2 mm, the width of the base about 125 μm, the width of the stem about 60 μm, the height of the body from the bottom of the base to the top of the stem about 15 μm, and the height of the base from the bottom of the base to the top of the base about 5 μm, with 16 electrode sites. Each of these was inserted into the zero-insertion force (ZIF) connector of a printed circuit board (PCB) containing linkages to each electrode trace in the MEA.

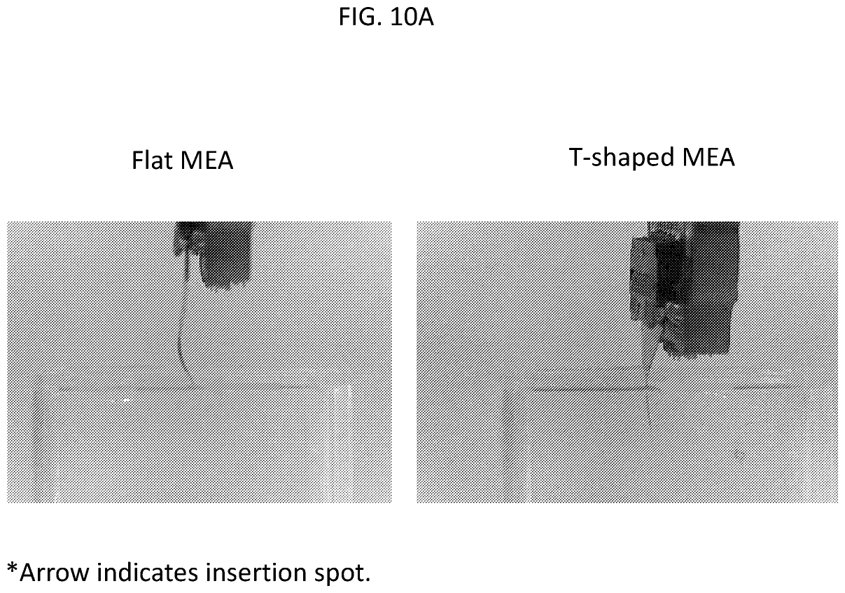

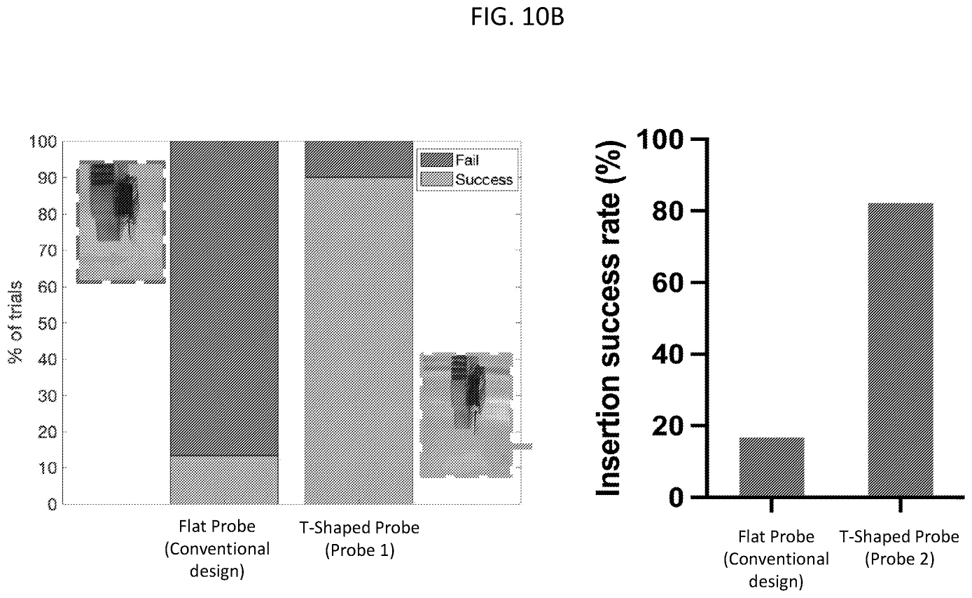

A 0.6% agarose gel was used as the brain tissue phantom. The PCB containing the MEA was attached to a stereotaxic arm whose speed was controlled by a piezo motor. To test the insertion mechanics in vitro, the MEA was inserted into the brain phantom by lowering the stereotaxic arm at the speed of 0.05 mm/s to the insertion depth of 2 mm. The test was performed 10 times for each electrode using three electrodes for each design (T-shaped and flat/standard). The success or failure of the MEAs for insertion into the gel without buckling was determined over a series of trials. FIG. 10A shows images of the flat MEA buckled during insertion, and the T-shaped MEA (Probe 1) inserted into the gel. For the Probe 1 assessment, the standard or flat design inserted only 13.3% of the trials while T shaped probe inserted successfully in 90% of the trials exhibiting 6.75-fold increase in success rate (FIG. 10B). For the Probe 2 assessment, the standard or flat design inserted only 18% of the trials while T shaped probe inserted successfully in 82% of the trials exhibiting 4.5-fold increase in success rate (FIG. 10B).

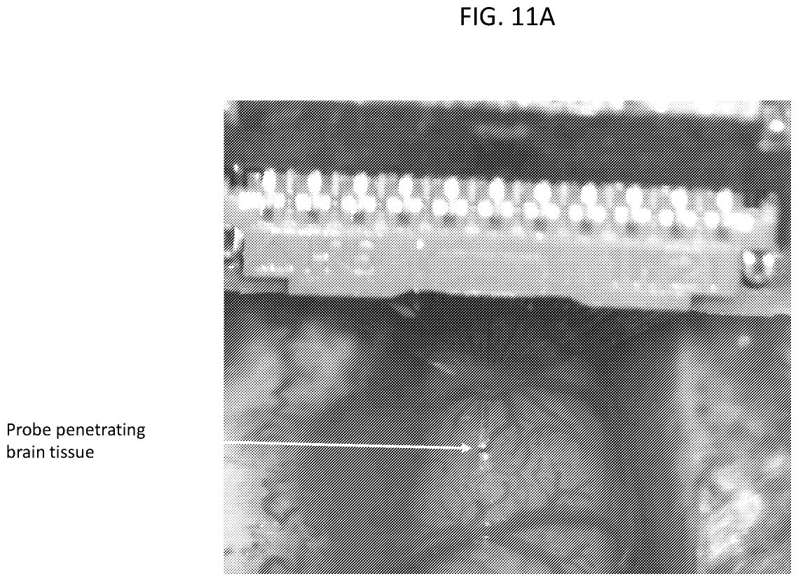

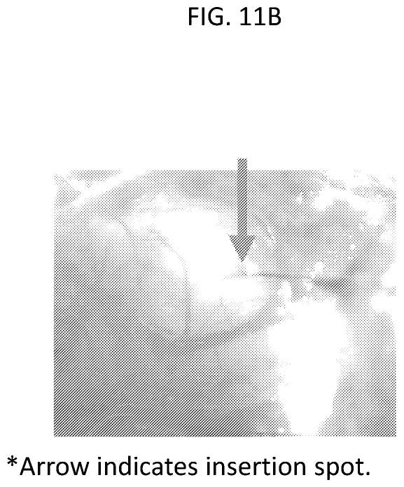

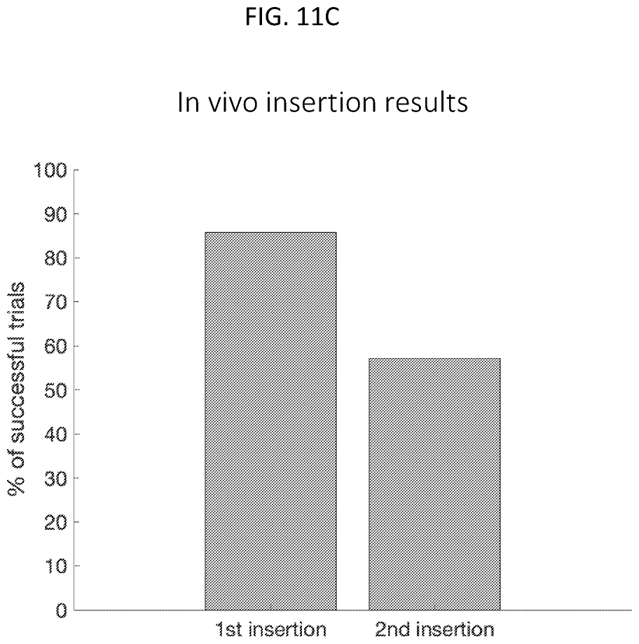

After validating that the T-beam MEA can insert into a brain phantom, similar insertion studies with the Probe 1 design were performed for the T-beam MEA in rats (n=3). Three male Sprague-Dawley rats (Charles River) were utilized to evaluate the in vivo insertion success of the T-beam MEA. The rats were anesthetized with isoflurane and head-fixed in a stereotaxic frame. The hair on the scalp was removed and a midline incision was performed. After retracting the scalp and clearing the blood and connective tissues between the scalp and skull, craniotomy was performed, and dura mater was carefully removed. The MEA was inserted into the zero-insertion force (ZIF) connector of a printed circuit board (PCB) containing linkages to each electrode trace in the MEA, as above. The PCB containing the MEA was attached to a stereotaxic arm whose speed was controlled by a piezo motor. The electrode was inserted into the brain to the depth of 1.5 mm. Insertion spots were chosen where there were no visible major blood vessels within the cranial window. FIGS. 11A and 11B show insertion of the MEA into the rat brain. Seven MEAs according to the Probe 1 dimensions were assessed. Each MEA was inserted and retracted repeatedly; the results for the first two insertions are shown in FIG. 11C.

It will be apparent that the precise details of the methods or compositions described may be varied or modified without departing from the spirit of the described aspects of the disclosure. We claim all such modifications and variations that fall within the scope and spirit of the claims below.

1. A penetrative intracortical microelectrode array (MEA), comprising:
a body having a substantially T-shaped cross-sectional area comprising a base and a stem, the body further comprising a proximal end and a distal end, and a longitudinal axis extending perpendicularly from the cross-sectional plane from the proximal end to the distal end, and wherein:
the base comprises a top and a bottom, with the stem extending from the top of the base,
the width of the base is greater than the width of the stem, with flanges of the base extending beyond either side of the stem;
the body is formed from an insulating biocompatible polymer surrounding individually addressable electrode traces connected to individually addressable electrode sites exposed to the exterior of the MEA;
the electrode traces are located within the stem of the MEA;
the electrode sites are located on the top of the flanges of the base;
the distal end is shaped for insertion into brain tissue and the proximal end is configured for connection to, or is connected to, an insertion device and also for electrical connection to an external neurostimulator or recording device for stimulating or recording neural signals from the electrode sites.

2. The MEA of claim 1, wherein the insulating biocompatible polymer is selected from parylene C, polyimide, and SU-8.

3. The MEA of claim 1, wherein the electrode traces and electrode sites are made of gold, platinum, iridium, indium tin oxide, conducting carbon, conducting polymer, or poly(3,4-ethylenedioxythiophene) (PEDOT).

4. The MEA of claim 1, wherein the distal end of the body has a pointed shape to facilitate penetration into cortical tissue.

5. The MEA of claim 1, comprising from 6 to 128 individually addressable electrode sites.

6. The MEA of claim 5, comprising 8, 16, or 32 individually addressable electrode sites.

7. The MEA of claim 1, wherein the electrode sites individually have a surface area of from 100-10,000 μm².

8. The MEA of claim 1, wherein the electrode sites are evenly distributed on each flange of the base.

9. The MEA of claim 1, wherein the electrode sites are evenly distributed within the distal half of the MEA and on the top of the flanges of the base.

10. The MEA of claim 1, wherein
the length of the body along the longitudinal axis is from 2-10 mm;
the width of the base is from 30-140 μm;
the width of the stem is from 15-60 μm;
the height of the body from the bottom of the base to the top of the stem is from 14-42 μm; and
the height of the base from the bottom of the base to the top of the base is from 5-14 μm.

11. The MEA of claim 10, wherein
(A)
the length of the body along the longitudinal axis is about 2 mm;
the width of the base is about 130 μm;
the width of the stem is about 50 μm;
the height of the body from the bottom of the base to the top of the stem is about 20 μm; and
the height of the base from the bottom of the base to the top of the base is about 8 μm; or
(B)
the length of the body along the longitudinal axis is about 2 mm;
the width of the base is about 125 μm;
the width of the stem is about 60 μm;
the height of the body from the bottom of the base to the top of the stem is about 15 μm; and
the height of the base from the bottom of the base to the top of the base is about 5 μm.

12. The MEA of claim 1, wherein the stem is centered on the base such that the flanges of the base extending beyond either side of the stem are about the same width.

13. The MEA of claim 1, wherein the MEA does not buckle when inserted into brain tissue with a force of 1 mN or less.

14. The MEA of claim 1, wherein MEA is not supported by a secondary solid substrate.

15. The MEA of claim 1, wherein the MEA is insertable into brain tissue without assistance of a shuttle, a secondary stiff coating, or an insertion device that supports the MEA from buckling during insertion.

16. A neural probe, comprising at least one MEA of claim 1.

17. A method of manufacturing the MEA of claim 1, comprising:
coating a SiO₂ surface of a silicon wafer with a first layer of the insulating biocompatible polymer, masking the first layer of insulating biocompatible polymer with a positive photoresist, aluminum, or alumina in the shape of the base of the body of the MEA via lithography, and etching the masked first layer of insulating biocompatible polymer to form a first layer of the base;
masking the first layer of the base with a positive photoresist in a pattern outlining the electrode sites and electrode connections between traces and electrode sites, photo-patterning and developing the masked first layer of the base, depositing a layer of conductive metal onto the photo-developed masked first layer of the base by metal evaporation, then stripping the positive photoresist to form a conductive metal containing first layer of the base;
coating the conductive metal containing first layer of the base with a second layer of the insulating biocompatible polymer, masking the second layer of insulating biocompatible polymer with a positive photoresist, aluminum, or alumina, in a pattern outlining the shape of the base with electrode sites, and photo-developing and etching the masked second layer to form the base;
repeating the coating, masking, photo-developing, depositing, and stripping steps to form the stem of the MEA containing electrode traces connected to the electrode sites in the base.
